(19) United States
(12) Patent Application Publication
(10) Pub. No.: US 2019/0328995 A1
(43) Pub. Date: Oct. 31, 2019

(54) AROMATHERAPY ESSENTIAL OIL COMPOSITIONS FOR REDUCING ANXIETY

(71) Applicant: Selena Irene Glenn, Portland, OR (US)

(72) Inventor: Selena Irene Glenn, Portland, OR (US)

(21) Appl. No.: 16/392,549

(22) Filed: Apr. 23, 2019

Related U.S. Application Data
(62) Division of application No. 14/993,279, filed on Jan. 12, 2016, now abandoned.
(60) Provisional application No. 62/103,740, filed on Jan. 15, 2015.

(51) Int. Cl. A61M 21/02; A61B 5/055

(52) U.S. Cl. CPC A61M 21/02; A61B 5/0555; A61M 2021/0016; A61M 2230/06; A61M 2205/59; A61M 2230/205

(57) ABSTRACT

A method to reduce anxiety and emotional stress includes one or a combination of aromatherapy essential oil blends. The blends include combinations which are fruity, floral, earth, and minty.

### CROSS-REFERENCE TO RELATED APPLICATIONS

This is a divisional of application No. 14/993,279, filed Jan. 12, 2016 with title “Method of complementary alternative medicine for mri anxiety”, provisional application No. 62/103,740 filed on Jan. 15, 2015 with the same title and naming Selena I. Glenn as inventor the entire content of which is hereby incorporated herein by reference.

### SUMMARY OF THE INVENTION

The present invention relates to a method and treatment for reducing anxiety of humans. More specifically, the present invention relates to aromatherapy, including the use of essential oils to reduce emotional stress.

In one embodiment of the invention, the method may include providing at least one aromatherapy scent The method may include placing at least one drop of the aromatherapy scent on a gauze. The aromatherapy scent may be a blend of two or more aromatherapy scents.

### BACKGROUND OF THE INVENTION

Despite advances in stress reduction, there remains yet a need for improved treatments. Further, there is a need for low-cost, solutions for this issue.

Accordingly, there remains a need for complementary and/or alternative medicine modalities, such as aromatherapy to reduce anxiety and reestablish emotional and physical homeostasis.

There has been extensive research found on anxiety. This literature review includes a general discussion of anxiety physiology, the common anxiety disorders along with of the specific physiology associated with these anxiety conditions. An in depth discussion of the literature about the correlations between aromatherapy and cognitive restructuring is included.

The physiology associated with anxiety primarily takes place in the area of the brain that controls our emotions, called the limbic system (Root, 2000). The limbic system controls our emotions through biochemical reactions regulated by a group of structures that intercommunicates with one another (Brown & Gerbarg, 2005a; Root, 2000; Starcevic, 2004). These structures include the prefrontal cortex, hippocampus, thalamus, periaquital grey matter, pineal gland, the olfactory region and amygdala (Root, 2000; Starcevic, 2004). Three structures in the limbic system, the prefrontal cortex, olfactory region and amygdala play a significant role in this thesis due to their regulatory effect on anxiety (Root, 2000; Starcevic, 2004).

The amygdala and its internal structures' role in emotion regulation are significant as they control two major branches of the autonomic nervous system, which are the parasympathetic and sympathetic nervous system (Root, 10 2000; Starcevic, 2004). These branches of the nervous system primarily govern the involuntary bodily functions such as blood pressure and heart rate (Blessing & Gibbins, 2008). A structure located within the amygdala, called the central nucleus controls emotions by regulating the heart and breathing rates, and blood pressure, and are major players in mood regulation (Root, 2000; Starcevic, 2004). Furthermore the central nucleus is the area of the brain anti-anxiety medications target to reduce panic attack symptoms, making it an significant player in anxiety regulation (Root, 2000).

The amygdala may contribute to anxiety as it is responsible for receiving and interrupting visual, and auditory stimulus and determines if an environment is safe or unsafe (Filley, 2011). The amygdala's role in anxiety is clear as it is one of the main limbic structures associated with anxiety disorders and responsible for interpreting threatening sounds (Filley, 2011). This correlation is supported by a study that determined the loud noise of MRI machines as a contributing factor in patient stress (Enders et al., 2011; Filley, 2011; McNulty & McNulty, 2009). The amygdala has direct connections between other parts of the limbic system that regulates emotions, such as the hippocampus, thalamus, periaquital grey matter, and prefrontal cortex (Starcevic, 2004).

The prefrontal cortex is regulated by a branch of the autonomic nervous system and regulates the brains reactions to dangerous situations (Brantley, 2007). The autonomic nervous system consists of two major branches, the sympathetic nervous system (SNS) and parasympathetic nervous system (PNS) (Blessing & Gibbins, 2008; Brantley, 2007). The PNS is responsible for activating the body's relaxation response, while the SNS prepares the body for action when under a perceived or real threat, often referred to as the fight-or-flight reaction (Blessing & Gibbins, 2008; Brantley, 2007). The prefrontal cortex is responsible for turning off the fight-or-flight mechanism of the sympathetic nervous system, which is important when a patient perceives an MRI as a threat (Brantley, 2007; Brown & Gerbarg, 2012).

The olfactory region of the limbic system plays a major role in anxiety regulation. Although the olfactory portion of the limbic system plays an important role in this thesis due to aromatherapy being a major topic, this will be discussed in greater detail in the aromatherapy section of this chapter.

The limbic system produces and regulates the biochemical reactions that control emotions (Blessing & Gibbins, 2008). Neurotransmitters are a result of these biochemical reactions that governs emotions such as anxiety (Root, 2000; Starcevic, 2004). Neurotransmitters essentially act as communicators by sending messages throughout the body via neuron synapses (Root, 2000). The neurotransmitters serotonin and gama aminobutyric acid, (GABA) are produced by the pineal gland and have inhibitory effects on anxiety. This inhibitory effect triggers a part of the PNS responsible for the relaxation response in the body, and in turn calms the fight or flight response when danger is perceived (Root, 2000; Starcevic, 2004). The neurotransmitters serotonin and GABA play an important role in emotions as low levels of either contributes to anxious feelings (Root, 2000). Norepinephrine is another neurotransmitter involved in emotions and is produced by the adrenal gland (Root, 2000). Norepinephrine has the opposite effect of serotonin and GABA, as it keeps the body alert and ready to take action when under threat, thus when it is overproduced it can cause anxiety (Root, 2000).

The two primary anxiety disorders are claustrophobia and panic attacks (Grey, Price, & Mathews, 2000; Sarji et al., 1998; Starcevic, 2004).

Claustrophobia: Claustrophobia defined as an extreme fear of small enclosed spaces (Grey et al., 2000; Sarji et al., 1998; Starcevic, 2004). A person who suffers from claustrophobia fears they will become trapped in an enclosed space and are accompanied by catastrophic thoughts or cognitions, which cause them to panic (Starcevic, 2004). Once a person begins to panic they begin to experience physiological symptoms such a shortness of breath, which is the most prominent symptom associated with claustrophobia (Craske et al., 2006; Starcevic, 2004). When a person experiences respiratory distress they begin to have cognitions that they will “suffocate and die” (Starcevic, 2004, p. 192), which is what happens when patients are in the MRI unit (Craske, Antony, & Barlow, 2006). This fear of suffocating may cause the patient to feel they are suffering from “air hunger” (Starcevic, 2004, p. 198), which is when the body gives a false alarm that it isn't receiving enough oxygen (Starcevic, 2004). The person then begins to breathe faster, and can lead to hyperventilation and ultimately a panic attack (Root, 2000; Starcevic, 2004).

Panic Attacks: A panic attack is characterized as an intense anxiety “episode” (Root, 2000, p. 2) that seemingly comes on without warning. Panic attacks reach climax in a matter of minutes, and generally last between (10) ten to (20) twenty minutes, but can last for an extended period of time (Root, 2000). Although the duration of panic attacks are relatively short, symptoms are perceived as lasting considerably longer to the person experiencing them (Root, 2000). Panic attacks are often experienced by people suffering from a variety of psychiatric disorders including phobias, and in some cases develop phobias due to having a panic attack (Root, 2000; Starcevic, 2004). There are three types of panic disorders including; 1. unexpected, which are those that happen without warning, 2. situational bound that always manifest in a specific situation, and 3. situationally predisposed which is when the likelihood of experiencing an anxiety episode increases in certain situations (Root, 2000; Starcevic, 2004).

The symptoms associated with a panic attack are considered either primary or secondary signs and are categorized by the frequency in which they occur (Starcevic, 2004). Primary symptoms include, a racing heart and/or heart palpitations, shortness of breath, a chocking feeling, uncontrollable shaking, sweating and dizziness, while secondary symptoms include nausea, hot and/or cold flashes, and finally tingling or numbness in extremities (Root, 2000; Starcevic, 2004). These physiological symptoms are governed by the autonomic nervous system, specifically the central nucleus of the amygdala, and the person is unable to control them. Although people have no control over the physiological symptoms some have successfully used slow breathing to influence reducing them (Starcevic, 2004). Furthermore, similar to claustrophobia, the onset of panic attack symptoms often causes catastrophizing thoughts, which exacerbates anxiety (Root, 2000; Starcevic, 2004).

When a person has a panic attack there is an overwhelming urge to react and leave the situation, thus patients knowing they can escape the situation can mean the difference between them feeling safe or unsafe in the environment (Root, 2000; Starcevic, 2004).

In conclusion, claustrophobia and panic attacks are major contributors to anxiety (Harris, Robinson, & Menzies, 1999; Thorpe et al., 2008; Tischler, Calton, Williams, & Cheetham, 2008).

Complementary alternative medicine has become increasingly popular in American culture and around the world (Chu & Wallis, 2007; Kessler et al., 2001; Lee, Wu, Tsang, Leung, & Cheung, 2011). An estimated 70% of Australians use CAM, in the UK just over 46% of the population have used it, while between 51% to 82% Taiwanese uses CAM (Chu & Wallis, 2007; Cooke, Mitchell, Tiralongo, & Murfield, 2012). In the USA approximately 40% of Americans have used some form of CAM in their health care with millions of out-of-pocket dollars spent on these therapies (Carrol, 2010). The popularity and financial lucrativeness of CAM has not gone unnoticed by conventional orthodox medicine practitioners, who have begun incorporating it into their practices (Astin, Marie, Pelletier, Hansen, & Haskell, 1998). A literature review of doctors showed there is a considerable number who either practiced CAM techniques with their patients or referred them to CAM practitioners (Astin et al., 1998). Nurses have also begun incorporating CAM into their practices, as a survey of critical care nurses concluded its use was commonly seen as a positive for their patients, with 93% who said it was helpful in easing anxiety (Cooke et al., 2012). Complementary medicine has been shown to be an effective and a popular way of treating anxiety (Kessler et al., 2001). A survey study by Kessler et al (2001) showed CAM to be a common way of treating anxiety amongst Americans. The study was conducted from 1997-1998 with a total of 2,055 participant assessing the incidence of CAM use by adult Americans and found 56% of those surveyed used CAM for anxiety (Kessler et al., 2001). This survey also showed those who used CAM for anxiety declared the therapies were just as effective in treating anxiety than conventional medicine (Kessler et al., 2001). Another study conducted by the Centers for Disease Control (CDS) in 2002 found that over 62% of participants used CAM, with anxiety being amongst the most popular ailment treated (Barnes, Powell-Griner, McFann, & Nahin, 2004). The growing acceptance of CAM and its gradual integration into conventional orthodox medicine (COM) along with research supporting its effectiveness in reducing anxiety reinforces its usefulness as an intervention (Carrol, 2010; Kessler et al., 2001; Wolpe, 2002).

Aromatherapy is a cognitive based interventions with immediate effects (Masaoka, Sugiyama, Katayama, Kashiwagi, & Homma, 2012). Aromatherapy can be described as a cognitive intervention, because it is a distraction technique and can help aide in changing cognitions, as scent has been shown to conjure memories (Redd et al., 1994; Zucco, Aiell, Turuani, & Koster, 2012). Furthermore aromatherapy has a direct links to our the limbic system which governs our cognitions (Brown & Gerbarg, 2005a, 2012; Masaoka et al., 2012).

Aromatherapy has been one of the most popular CAM therapies used to help reduce anxiety (Hongratanaworakit, 2004; Lee et al., 2011; Perry & Perry, 2006). Aromatherapy, defined as using essential oils for therapeutic purposes either through dermal or inhalation administration (Pease & Pease, 2004). Please note all studies used in this literature review used inhaled aromatherapy as this was the route used in the pilot study. Furthermore, inhalation is believed to be the primary healing source aromatherapy has on emotions (Price & Price, 2007).

An advantage of using aromatherapy, is the immediate physiological and psychological effects it has when compared to other currently used interventions such as sedation, or hypnosis (Perry & Perry, 2006; Meledez & McCrank, 1993). Inhaled aromatherapy's immediate effect is due to the scent molecules ability to bypass the blood brain barrier a go directly to the olfactory portions of the limbic system, which regulates emotions via the autonomic nervous system (ANS) (Homma & Masaoka, 2008; Hongratanaworakit, 2004; Perry & Perry, 2006; Root, 2000). When a scent molecule enters the nasal cavity it attaches to the olfactory epithelial receptors. These receptors take this scent information and travels along the olfactory nerves and signals the limbic system via the hippocampus within the amygdala (Jimbo, Kimura, Taniguchi, Inoue, & Urakami, 2009). Once this information has reached the hippocam pus the ANS is activated, neurotransmitters are secreted. The type of neurotransmitter secreted depends on which branch of the ANS is activated. If the odor is pleasant and/or attached to positive memories the parasympathetic nervous system (PNS) is activated and neurotransmitters such as GABA and serotonin are secreted producing a relaxation effect (Jimbo et al., 2009; Root, 2000; Zucco et al., 2012). In the case of an unpleasant scent and/or negative emotions attached to it the sympathetic nervous system (SNS) is activated and neurotransmitters such as norepinephrine are secreted creating anxious feelings (Jimbo et al., 2009; Root, 2000; Zucco et al., 2012). This action takes place in less than a second, approximately “300 ms-400 ms” (Homma & Masaoka, 2008, p. 1016) and reaction time may be dependent on gender with women having faster emotional reactions then men (Chen & Dalton, 2005; Perry & Perry, 2006). In short aromatherapy is the result of the scent molecule interacting with the limbic system and is dependent on the respiratory system (Homma & Masaoka, 2008; Jimbo et al., 2009).

Using a blend of essential oils may be more effective in reducing anxiety then single oils (Burns, Blamey, Ersser, Barnetson, & Lloyd, 2000). Studies have shown aromatherapy to be effective at lowering anxiety; however the majority of these used single oils in their interventions creating a gap in the literature (Burns et al., 2000). Several single essential oils have been studied in reducing to including, but not limited to lavender, rosemary, bergamot, ylang ylang, rosemary, melissa, valerian, roman chamomile, neroli, cedarwood, orange oil, eucalyptus, rose, jasmine, and vetiver (Gorji, Koulivand, & Ghadiri, 2013; Lee et al., 2011; Moss, Cook, Wesnes, & Duckett, 2003; Saiyudthong & Marsden, 15 2011).

Aromatherapy has been used to help reduce anxiety in a variety of medical settings. There have been several studies evaluating its use in reducing anxiety dental, post-surgical, and postpartum anxiety with positive result (Braden, Reichow, & Halm, 2009; Lee et al., 2011; Lehrner, Marwinski, Lehr, Johren, & Deecke, 2005). A pilot study by Conrad & Adams (2012) evaluating the effects lavender on anxiety and depression of postpartum women found a significant reduction of anxiety in the experimental using the aromatherapy. Although this study showed positive results, only women were used, which limited the verifiable effect to women (Conrad & Adams, 2012). Another study reviewing the effect lavender had on pain and anxiety of post-operative patients had an equal number of male and female participants, and showed a significant reduction of both pain and anxiety supporting the effectiveness of aromatherapy for both genders (Braden et al., 2009).

Other studies demonstrated various medical procedures aromatherapy has been used, included using essential oils to reduce dental phobia in children. This study concluded aromatherapy helped reduce dental procedure fears in children (Jafarzadeh, Arman, & Pour, 2013). Another study assessing the effect vaporized orange oil had on dental patients showed increase activity of the parasympathetic nervous (PNS), and promoted relaxation by 12% while decreasing the sympathetic nervous system (SNS) by 16% and showed a significant reduction on anxiety in adult dental patients (Jafarzadeh et al., 2013; Lehrner et al., 2005; Perry & Perry, 2006). While this study was conducted on both men and women, anxiety reduction was more pronounced in women (Lehrner et al., 2005).

A systematic literature review of studies that used aromatherapy to reduce anxiety over a twenty year period was conducted. Out of the 14 studies reviewed six (6) used inhaled aromatherapy as an intervention with one using music therapy as well (Lee et al., 2011). Five of the six studies showed aromatherapy to be effective in reducing anxiety and one showing no significant change (Lee et al., 2011). These studies included participants that experienced anxiety for a variety of reasons, with the majority undergoing medical treatments and a diverse group of individuals. This literature review concluded aromatherapy could in fact be used to help reduce the symptoms of anxiety in a variety of medical environments (Lee et al., 2011).

Aromatherapy was reported to help treat anxiety, including cognitive and stress related disorders (Perry & Perry, 2006; Conrad & Adams, 2012).

The use of fragrance has been successfully used to reduce anxiety in the MRI environment (Redd et al., 1994). Two studies dealing specifically with using scents in the MRI environment was found. The earlier study conducted by Redd et al (1994) showed a reduction in overall MRI anxiety by 63% in patients using a vanilla scent administered through a nasal cannula (Redd et al., 1994). This study went one step further and evaluated the experimental group participants who considered the scent pleasing and their level of anxiety and found a noteworthy reduction in those who found the aroma pleasant (Redd et al., 1994). This study hypothesized fragrance had a cognitive effect on patients enabling them to remain calm during their examination. This was in alignment with a study that concluded scents can affect a person's mood and consequently their cognitions (Perry & Perry, 2006; Redd et al., 1994). In essence the scent may have blocked anxious feelings and replaced them with more positive ones (Redd et al., 1994). Furthermore a pleasant scent causes people to slow their breath and deepen their inhalations triggering a relaxation response (Moore, Hickson, & Stacks, 2010). This same study noted possibly decreasing anxiety further in MRI patients by allowing them to choose their own scent and adding relaxations techniques (Redd et al., 1994). This pilot study used this suggestion and allowed patients to choose their own scent and incorporated breathing exercises as an additional relaxation technique. Lastly this study concluded that while positive results were shown in using scent to reduce MRI anxiety, others studies reproducing its results will be necessary before using fragrance can be include as a standard of care in controlling MRI anxiety (Redd et al., 1994).

Scent can increase a customer's sense of satisfaction (Goldkuhl & Styven, 2007). Scents have been shown to elicit strong emotional reactions with anxious individuals being more responsive to pleasant aromas, making it a feasible way of decreasing anxiety (Chen & Dalton, 2005; Goldkuhl & Styvén, 2007; Zucco et al., 2012).

### DETAILED DESCRIPTION OF PREFERRED EMBODIMENTS

Certain preferred embodiments of a method according to the present invention include the following aromatherapy essential oils alone, or in any combination.

This invention shows how complementary and/or alternative medicine modality aromatherapy can be effective in reducing anxiety and reestablishing emotional and physical homeostasis.

The four different aromatherapy scent blends include pure undiluted essential oils with the exception of vanilla, which was a CO2 extract, and administered inactively through inhalation. The blends include; #1 a fruity, #2 floral, #3 earthy, and #4 minty. The essential oils used include bergamot, lemongrass, spearmint, rosemary, cardamom, oil of orange, vetiver, geranium, lavender, patchouli, sandalwood, palmarosa, roman chamomile, pettigraine, neroli, vanilla CO2, ylang ylang and marjoram.

The following are formulations of each aromatherapy blend; these blends are approximately one (1) fluid ounce each. These are the quantities as they were used in the study. The origins of the aromatherapy scents are provided in parentheses. In other embodiments of the invention or formulations, the scents have different origins.

Fruity Blend (Approx.) 1.10 Z: Orange 5 fold (USA)=9.5 ml, Lemongrass (Indian)=5 ml, Vanilla CO2=8.5 ml, Neroli (Egyptian)=2.5 ml, Bergamot (Italian)=5 ml, and Pettigraine (Paraguay)=2.5 ml.

Floral Blend (Approx.) 10 Z, Total of 30.08 ml: Palmarosa (India)=7.5 ml, Geranium (Egypt)=5.65 ml, Roman Chamomile (Oregon)=1.87 ml, Lavender (Oregon)=3.76 ml, Ylang Ylang 2nd (Madagascar)=5.65 ml, and Vanilla CO2=5.65 ml.

Earthy Blend (Approx.) 10 Z: Patchouli Dark Aged (Indonesia)=5.25 ml, Sandalwood Mysore (India)=2.6 ml, Vanilla CO2 =1.05 ml, Lemongrass (India)=1.6 ml, Cardamom Whole (India)=1.6 ml, Orange 5 Fold (USA)=1.6 ml, Vetiver (Indonesia)=3.7 ml, Geranium (Egypt)=10.5 ml, and Bergamot (Italian)=2.1 ml.

Minty Blend (Approx.) 10 Z: Rosemary Verbenone (USA)=5 ml, Spearmint (Oregon)=12.5 ml, and Marjoram Sweet (Hungary)=12.5 ml.

The versatility of possible scents can be adapted to fit any ethnic group or nationality, broadening its global reach because its adaptability is the key to this technique having a global impact. Different cultures have diverse associations with particular scents. For example lavender is associated with death in certain countries and may not be effective in reducing anxiety, whereas in others the associations are more positive (Lawless, 1994).

The relevance of the social context is clear in that it helps reduces anxiety. This includes providing an alternative to taking narcotics for anxiety, and those who cannot take sedatives due to allergies and sobriety issues.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A plant essential oil composition for nasal administration consisting of at least one or more essential oils selected from: orange 5 fold, lemongrass, vanilla CO2, neroli, bergamot, pettigraine, palmarosa, geranium, roman chamomile, lavender, ylang ylang, patchouli, sandalwood, cardamom, vetiver, geranium, rosemary verbenone, spearmint, and marjoram sweet.

2. The plant essential oil composition of claim 1, wherein the essential oils comprises of;
Fruity Blend approximately 1.1 0 Z: Orange 5 fold˜9.5 ml, Lemongrass˜5 ml, Vanilla CO2˜8.5 ml, Neroli˜2.5 ml, Bergamot˜5 ml, and Pettigraine˜2.5 ml.

3. The plant essential oil composition of claim 1, wherein the essential oils comprises of;
Floral Blend approximately 1.0 0 Z: Palmarosa˜7.5 ml, Geranium˜5.65 ml, Roman Chamomile˜1.87 ml, Lavender˜3.76 ml, Ylang Ylang˜5.65 ml, and Vanilla CO2˜5.65 ml.

4. The plant essential oil composition of claim 1, wherein the essential oils comprises of;
Earthy Blend approximately 1.0 0 Z: Patchouli˜5.25 ml, Sandalwood Mysore˜2.6 ml, Vanilla CO2˜1.05 ml, Lemongrass˜1.6 ml, Cardamom Whole˜1.6 ml, Orange 5 Fold˜1.6 ml, Vetiver˜3.7 ml, Geranium˜10.5 ml, and Bergamot˜2.1 ml.

5. The plant essential oil composition of claim 1, wherein the essential oils comprises of;
Minty Blend approximately 1.0 0 Z: Rosemary Verbenone˜5 ml, Spearmint˜12.5 ml, and Marjoram Sweet˜12.5 ml.

6. A plant essential oil composition for treating anxiety and emotional stress consisting of at least one or more essential oils selected from: orange 5 fold, lemongrass, vanilla CO2, neroli, bergamot, pettigraine, palmarosa, geranium, roman chamomile, lavender, ylang ylang, patchouli, sandalwood, cardamom, vetiver, geranium, rosemary verbenone, spearmint, and marjoram sweet.

7. The plant essential oil composition of claim 6, wherein the essential oils comprises of;
Fruity Blend approximately 1.1 0 Z: Orange 5 fold˜9.5 ml, Lemongrass˜5 ml, Vanilla CO2˜8.5 ml, Neroli˜2.5 ml, Bergamot˜5 ml, and Pettigraine˜2.5 ml.

8. The plant essential oil composition of claim 6, wherein the essential oils comprises of;
Floral Blend approximately 1.0 0 Z: Palmarosa˜7.5 ml, Geranium˜5.65 ml, Roman Chamomile˜1.87 ml, Lavender˜3.76 ml, Ylang Ylang˜5.65 ml, and Vanilla CO2˜5.65 ml.

9. The plant essential oil composition of claim 6, wherein the essential oils comprises of;
Earthy Blend approximately 1.0 0 Z: Patchouli˜5.25 ml, Sandalwood Mysore˜2.6 ml, Vanilla CO2˜1.05 ml, Lemongrass˜1.6 ml, Cardamom Whole˜1.6 ml, Orange 5 Fold˜1.6 ml, Vetiver˜3.7 ml, Geranium˜10.5 ml, and Bergamot˜2.1 ml.

10. The plant essential oil composition of claim 6, wherein the essential oils comprises of;
Minty Blend approximately 1.0 0 Z: Rosemary Verbenone˜5 ml, Spearmint˜12.5 ml, and Marjoram Sweet˜12.5 ml.

11. A method for the treatment of anxiety and emotional stress comprising the step of administering to a person in need thereof an effective amount of at least one or more essential oils selected from: orange 5 fold, lemongrass, vanilla CO2, neroli, bergamot, pettigraine, palmarosa, geranium, roman chamomile, lavender, ylang ylang , patchouli, sandalwood, cardamom, vetiver, geranium, rosemary verbenone, spearmint, and marjoram sweet.

12. The plant essential oil composition of claim 11, wherein the essential oils comprises of;
Fruity Blend approximately 1.1 0 Z: Orange 5 fold˜9.5 ml, Lemongrass 5 ml, Vanilla CO2˜8.5 ml, Neroli˜2.5 ml, Bergamot˜5 ml, and Pettigraine˜2.5 ml.

13. The plant essential oil composition of claim 11, wherein the essential oils comprises of;
Floral Blend approximately 1.0 0 Z: Palmarosa˜7.5 ml, Geranium˜5.65 ml, Roman Chamomile˜1.87 ml, Lavender˜3.76 ml, Ylang Ylang˜5.65 ml, and Vanilla CO2 ˜5.65 ml.

14. The plant essential oil composition of claim 11, wherein the essential oils comprises of;
Earthy Blend approximately 1.0 0 Z: Patchouli˜5.25 ml, Sandalwood Mysore˜2.6 ml, Vanilla CO2˜1.05 ml, Lemongrass˜1.6 ml, Cardamom Whole˜1.6 ml, Orange 5 Fold˜1.6 ml, Vetiver˜3.7 ml, Geranium˜10.5 ml, and Bergamot˜2.1 ml.

15. The plant essential oil composition of claim 11, wherein the essential oils comprises of;
Minty Blend approximately 1.0 0 Z: Rosemary Verbenone˜5 ml, Spearmint˜12.5 ml, and Marjoram Sweet˜12.5 ml.
